(19) United States
(12) Patent Application Publication
(10) Pub. No.: US 2016/0220719 A1
(43) Pub. Date: Aug. 4, 2016

(54) METHOD OF MAKING INTERIOR DECORATION MATERIAL, ACTIVE MATERIAL AND MANUFACTURING METHOD THEREOF

(71) Applicant: NINGBO CALLFO NEW MATERIAL SCIENCE & TECHNOLOGY CO., LTD., Cixi, Zheijiang Province (CN)

(72) Inventor: Huimin Cheng, Cixi, Zhejiang Province (CN)

(73) Assignee: NINGBO CALLFO NEW MATERIAL SCIENCE & TECHNOLOGY CO., LTD., Cixi, Zhejiang Province (CN)

(21) Appl. No.: 14/890,315

(22) PCT Filed: Apr. 28, 2014
(86) PCT No.: PCT/CN2014/076352

§ 371 Date: Nov. 10, 2015

(30) Foreign Application Priority Data
Oct. 21, 2013 (CN) 201310492027.2

(51) Int. Cl. A61L 9/01; B05D 3/00

(52) U.S. Cl. CPC A61L 9/01; B05D 3/007

(57) ABSTRACT

The invention discloses a method of making an interior decoration material, an active material and a manufacturing method thereof, and the active material can be used to make an article inside a car and a furniture, wherein the method of making the interior decoration material that can eliminate an odor and a harmful gas has an interior decoration active material that releases a far infrared ray having a wavelength of 5-15 and can ionize negative ions in the air, has a surface air ion concentration reaching 3000-15000/cc or more, and has a function of rapidly eliminating and decomposing the odor such as methanal, benzene a smell of smoke and the harmful gas, and is obtained by preparation by subjecting raw materials to blending and drying, and the respective constituent raw materials and contents thereof during the blending are as follows: 100 parts of resin, 5-30 parts of Maifan stone powder, 5-10 parts of quartz powder, 5-30 parts of tourmaline powder, and 1-5 parts of rare earth, and the mass part of the resin is taken as a standard.

### TECHNICAL FIELD

The invention relates to a decoration material, in particular, to a method of making an interior decoration material, an active material and a manufacturing method thereof.

### TECHNICAL BACKGROUND

After a new car is produced, a pungent odor will be firstly smelled as soon as the door of the car is opened. The Air Test Center of the China National Interior Decoration Association has ever performed a testing to 200 cars and produced unexpected results. Where an indoor air quality standard is referred to, a problem will exist that a content of methanal or benzene in the air inside the car is out of a limit in nearly 90% of cars, and in most of the cars the methanal is out of the limit by five or six times or more. Toxic chemical substances inside the cars presented by methanal and VOC may cause allergy and aggravate cough and asthma in a light way, and may even cause a change in a cell structure, a lesion of a nerve center or a cancer, and even cause death in a serious way. At present, the problem of air pollution inside a car has been clarified by the World Health Organization as one of ten major threats to the human health, and listed by the United States as one of five major damages to the human health.

Although an air cleaner inside a car can purify the air, but its result and not satisfied and what's more, ozone will be produced to cause damage to a human body. Therefore a technical solution of effective cleaning pollution inside a car is absent in prior arts.

Meanwhile, it is known to all that a method of decorating a surface of furniture has a nonnegligible effect on reduction of methanal. At present, either wood furniture or fabric furniture will produce massive harmful gases after they are made out. Because methanal has a function of fixing colors, adding of methanal in a dyestuff or an assistant is for the purpose of preventing colors' fading. In addition, an adhesive, various decoration materials, and painting or coating materials themselves also contain a large amount of methanal and other harmful substances. How to effectively control release of toxic and harmful gases after a material of furniture is selected and finished furniture is made is a hot problem in the current whole furniture and building materials market, and is also a problem to which consumers pay attention.

### SUMMARY

The main object of the invention is to provide a method of making an interior decoration material, an active material and a manufacturing method thereof to resolve a problem that toxic and harmful substances cannot be continuously eliminated in some key areas and environments in prior arts, wherein:

The embodiment of the invention provides a method of making an interior decoration material that can eliminate odors and harmful gases, characterized in that: an interior decoration active material that releases a far infrared ray having a wavelength of 5-15 and can ionize negative ions in the air is included, said interior decoration material has a surface air ion concentration reaching 3000-15000/cc or more, has a function of rapidly eliminating and decomposing the odor such as methanal, benzene and a smell of smoke and the harmful gas, and is obtained by preparation by subjecting raw materials to blending and drying, the respective constituent raw materials and contents thereof during the blending are as follows: 100 parts of resin, 5-30 parts of Maifan stone powder, 5-10 parts of quartz powder, 5-30 parts of tourmaline powder, and 1-5 parts of rare earth, and the mass part of the resin is taken as a standard.

Further, the Maifan stone powder, the quartz powder, the tourmaline powder and the rare earth are uniformly mixed, then added to a resin material to be stirred, defoamed and ground to form a slurry that is subsequently coated on a polyurethane material surface by a coating amount of 200 g/m2, and then dried inside a drying oven of a temperature of 100-130° C. to form a finished product.

The embodiment of the invention further provides an active material, which comprises a surface layer whose constituent raw materials and contents thereof include: 100 parts of resin, 5-30 parts of nano-size Maifan stone, 5-10 parts of nano-size quartz powder, 5-30 parts of nano-size tourmaline powder, and 1-5 parts of nano-size rare earth, wherein the mass part of the resin is taken as a standard; and a polyurethane bass.

Further, the constituent raw materials of said surface layer and the contents thereof include: 100 parts of resin, 5-8 parts of nano-size Maifan stone powder, 5-8 parts of nano-size quartz powder, 5-10 parts of nano-size tourmaline powder, and 1-5 parts of nano-size rare earth, wherein the mass part of the resin is taken as a standard.

Further, said polyurethane bass includes a polyurethane material and a fiber layer, or includes PVC and a fiber layer.

The embodiment of the invention further provides an article inside a car made of the active material according to any one of claims 3-5.

Further, said article inside a car comprises: a car floor mat, a car seat mat, a neck pillow, a throw pillow and a back pillow for a car, a car steering wheel cover, a car door trim cover, a roof inside a car, a car blanket, a car seat cover, a car safety belt, a package inside a trunk, a storage box for a car, a glove compartment inside a car, and an ornament inside a car.

In addition, the embodiment of the invention further provides a furniture made of the active material according to any one of claims 3-5.

The embodiment of the invention further provides a method of making an active material, comprising: uniformly stirring and defoaming predetermined constituent raw materials to form a surface layer, wherein said predetermined constituent raw materials and contents thereof include: 100 parts of resin, 5-30 parts of Maifan stone powder, 5-10 parts of quartz powder, 5-30 parts of tourmaline powder, and 1-5 parts of rare earth, wherein the mass part of the resin is taken as a standard; and coating the surface layer on a release paper, sticking a polyurethane bass after drying, and performing a separation from the release paper after drying and cooling.

Further, said predetermined constituent raw materials and the contents thereof include: 100 parts of resin, 5-8 parts of Maifan stone powder, 5-8 parts of quartz powder, 5-10 parts of tourmaline powder, and 1-5 parts of rare earth, wherein the mass part of the resin is taken as a standard.

According to the technical solution of the invention, the active material can effectively eliminate toxic chemical substances such as methanal and VOC inside a car or a room.

### BRIEF DESCRIPTION OF THE DRAWINGS

The figure illustrated herein is used for providing a further understanding of the invention, and forms a part of the application, and schematic embodiments of the invention and descriptions thereof are used for explaining the invention, and do not form improper limitations of the invention. In the Drawings:

FIG. 1 is a flow chart of a method of making an active material of an embodiment of the invention.

### DETAILED DESCRIPTION

In order to make the object, technical solution and advantages of the invention clearer, the figure and embodiments are taken into consideration to make further detailed descriptions of the invention below:

According to the embodiment of the invention, an active material is provided comprising: a surface layer and a polyurethane (PU) bass. Specifically, constituent raw materials of the surface layer and weight percentages thereof include: 100 parts of resin (a TPU material), 5-30 parts of nano-size Maifan, 5-10 parts of nano-size quartz powder, 5-30 parts of nano-size tourmaline powder, and 1-5 parts of nano-size rare earth, wherein the mass part of the resin is taken as a standard. The nanoscale Maifan stone is also called as nanoscale medical stone, medicinal stone or Maifanshi.

In one embodiment of the invention, said surface layer can also be constituted by following formulations: 100 parts of resin, 5-8 parts of nano-size Maifan stone powder, 5-8 parts of nano-size quartz powder, 5-10 parts of nano-size tourmaline powder, and 1-5 parts of nano-size rare earth, wherein the mass part of the resin is taken as a standard.

Said PU bass refers to a polyurethane material and a fiber cloth, or includes PVC and a fiber layer.

According to the embodiment of the invention, a car interior article made of the above-mentioned active material is further provided.

Further, the car interior article includes but is not limited to a car floor mat, a car seat mat, a neck pillow, a throw pillow and a back pillow for a car, a car steering wheel cover, a car door trim cover, a roof inside a car, a car blanket, a car seat cover, a car safety belt, a package inside a trunk, a storage box for a car, a glove compartment inside a car, and an ornament inside a car, etc.

According to the embodiment of the invention, a furniture product is further provided and the furniture can be either a home used furniture or an office used furniture, on which the active materials are laminated to the surface of the main material of the furniture for the purpose of obtaining healthy materials and continuously eliminating toxic and harmful substances. The furniture includes but is not limited to hardwood furniture, panel furniture, software furniture (such as fabric or leather furniture), steel-wood furniture, furniture made of other man-made materials and the like.

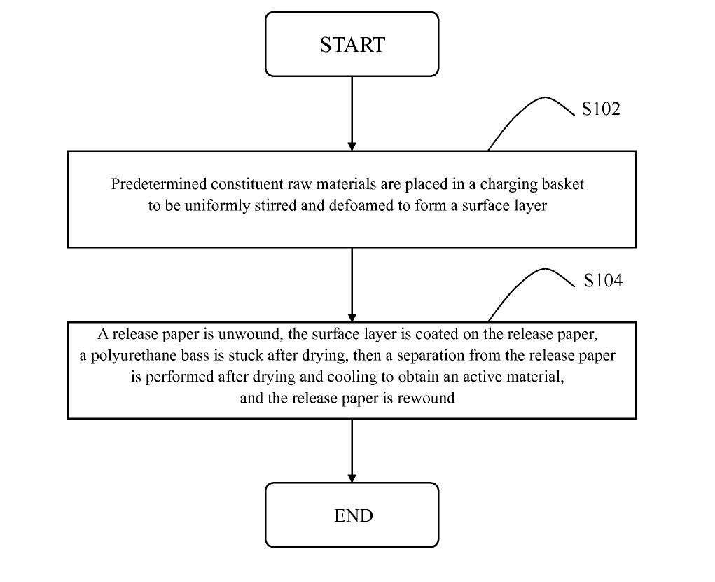

According to the embodiment of the invention, a method of making an active material is further provided, and referring to FIG. 1, the method comprises:

(1) Placing predetermined constituent raw materials in a charging basket, then forming a surface layer after stirring uniformly and defoaming.

In one embodiment, the predetermined constituent raw materials and weight percentages thereof include: 100 parts of resin, 5-30 parts of Maifan stone powder, 5-10 parts of quartz powder, 5-30 parts of tourmaline powder, and 1-5 parts of rare earth, wherein the mass part of the resin is taken as a standard.

In another embodiment, said predetermined constituent raw materials and weight percentages thereof include: 100 parts of resin, 5-8 parts of Maifan stone powder, 5-8 parts of quartz powder, 5-10 parts of tourmaline powder, and 1-5 parts of rare earth, wherein the mass part of the resin is taken as a standard.

(2) Unwinding a release paper, coating the surface layer on the release paper, sticking a polyurethane (PU) bass after drying, then performing a separation from the release paper after drying and cooling, then obtaining an active material, and rewinding the release paper.

Finally, after being unwound and with the procedures such as printing, embossing, slitting and packaging, the manufacture of the active material is completed.

Experiments show that settling the articles inside the car such as the car seat mat and the car throw pillow made of the active material provided by the invention inside the car, each of them can eliminate original smells inside the car within one week; the smell of smoke can be completely eliminated within 24 hours after smoking is done inside the car; both a driver and occupants can feel the fresh air inside the car, and fatigue is reduced; the national indoor standard can be reached through a methanal detection after 10 days from the installation; and space negative ions are detected by a KEC-900 negative ion concentration detector, and fluctuation value of an average negative ion concentration inside the car is 3,000/cc-20,000/cc.

According to the technical solution of the invention, the active material can effectively eliminate toxic chemical substances such as methanal and VOC inside a car or a room.

According to the embodiment of the invention, an interior decoration material that can eliminate odors and harmful gases is further provided, and is obtained by adding Maifan stone powder, quartz powder, tourmaline powder, rare earth and the like to PU resin. The Maifan stone powder, quartz powder, tourmaline powder, rare earth and the like are mixed, and 45 parts or less of said total mixed materials are added to 100 parts of resin. Specifically, firstly the Maifan stone powder, quartz powder, tourmaline powder, rare earth and the like are uniformly mixed, then added to a resin material to be stirred, defoamed and ground by a grinding device to form a slurry that is subsequently coated on a PVC leather or PU material surface, and then dried inside a drying oven of a temperature of 100-130° C. to form a finished product. The interior decoration active material is used for decoration in a car or a family room, which can release a far infrared ray having a wavelength of 5-15 um with 82% or above of an infrared ray release rate and can ionize negative ions in the air with 3,000-15,000/cc or more of a surface air ion concentration. What's more, it has a function of rapidly eliminating and decomposing odors such as methanal, benzene, a smell of smoke and other harmful gases. The material can be used for a decoration inside a car, a decoration inside a room of a house and a decoration of other space.

The interior decoration material which eliminates odors and harmful gases is capable of releasing a far infrared ray having a wavelength of 5-15 and ionizing negative ions in the air. By this way a surface air ion concentration can reach 3,000-15,000/cc or more. What's more the interior decoration material has a function of rapidly eliminating and decomposing odors such as methanal, benzene, a smell of smoke and other harmful gases. It is obtained according to the following raw materials and contents through preparation steps after blending and drying procedures: resin: 100 parts; Maifan stone powder: 5-30 parts; quartz powder: 5-10 parts; tourmaline powder: 2-30 parts; and rare earth and the like: 0.5-10 parts; and the mass part of the resin is taken as a standard.

The method of making the interior decoration material that eliminates odors and harmful gases is as follows: firstly uniformly mixing the Maifan stone powder, the quartz powder, the tourmaline powder, the rare earth and the like; then adding them to a resin material to be stirred, defoamed and ground by a grinding device to form a slurry that is subsequently coated on a PVC leather or PU leather material surface by a coating amount of 200 g/m2. After drying procedure in a drying oven with a temperature of 100-130° C., a finished product is formed.

In the interior decoration material that eliminates odors and harmful gases, the resin can be selected from a PU material, a PVC surface material, an acroleic acid mixed material etc. The Maifan stone is selected from a material with a mesh number of 1000-2000, and is added by an amount of 30 parts or less; the quartz powder is selected from a material having a mesh number of 4000 or more, and is added by an amount of 10 parts or less; the tourmaline powder is selected from a material with high purity having a mesh number of 2500 or more, and is added by an amount of 30 parts or less; and the rare earth is selected from a high purity material having a mesh number of 2500 or more, and is added by an amount of 10 parts or less.

The interior decoration material that eliminates odors and harmful gases has a capability to release an infrared ray. It can release a far infrared ray having a wavelength of 5-15 with an infrared ray release rate reaching 82% or more.

The interior decoration material that eliminates odors and harmful gases has a capability to ionize the surface air, and has a function of releasing negative ions.

The interior decoration material that eliminates odors and harmful gases has a function of rapidly eliminating odors such as methanal, benzene, a smell of smoke and the harmful gas.

The contents mentioned above are only embodiments of the invention and are not used for limiting the invention, and to those skilled in the art, the invention can be modified and changed in various manners. All amendments, equivalent substitutions, improvements and the like made within the spirit and principle of the invention should be contained within the scopes of the claims of the invention.

1. A method of making an interior decoration material that eliminates odors and harmful gases, by comprising:
providing raw materials by the following contents: 100 parts of resin, 5-30 parts of Maifan stone powder, 5-10 parts of quartz powder, 5-30 parts of tourmaline powder, and 1-5 parts of rare earth, and the mass part of the resin is taken as a standard;
blending and drying the raw materials;
obtaining an interior decoration active material that releases a far infrared ray having a wavelength of 5-15 and can ionize negative ions in the air, having a surface air ion concentration reaching 3000-15000/cc or more.

2. The method of making an interior decoration material that eliminates odors and harmful gases according to claim 1, by comprising:
uniformly mixing the Maifan stone powder, the quartz powder, the tourmaline powder and the rare earth;
then adding them to the resin to be stirred, defoamed and ground to form a slurry which is subsequently coated on a PVC material or a PU material surface by a coating amount of 200 g/m²; and
drying inside a drying oven with a temperature of 100-130° C. to form a finished product.

3. An active material used for an interior decoration, comprising:
a surface layer, which is provided by a formulation including: 100 parts of resin, 5-30 parts of nano-size Maifan stone, 5-10 parts of nano-size quartz powder, 5-30 parts of nano-size tourmaline powder, and 1-5 parts of nano-size rare earth, wherein the mass part of the resin is taken as a standard; and
a polyurethane bass.

4. The active material used for an interior decoration according to claim 3, wherein the surface layer is provided by a formulation including: 100 parts of resin, 5-8 parts of nano-size Maifan stone powder, 5-8 parts of nano-size quartz powder, 5-10 parts of nano-size tourmaline powder, and 1-5 parts of nano-size rare earth, wherein the mass part of the resin is taken as a standard.

5. The active material used for an interior decoration according to claim 3, wherein the polyurethane bass includes a polyurethane material and a fiber layer, or includes PVC and a fiber layer.

6. A car interior article, wherein said article is made using the active material used for an interior decoration according to claim 3.

7. The car interior article according to claim 6, comprising: a car floor mat, a car seat mat, a neck pillow, a throw pillow and a back pillow for a car, a car steering wheel cover, a car door trim cover, a roof inside a car, a car blanket, a car seat cover, a car safety belt, a package inside a trunk, a storage box for a car, a glove compartment inside a car, and an ornament inside a car.

8. A furniture, the furniture is made using the active material according to claim 3.

9. A method of making an active material used for an interior decoration, comprising:
uniformly stirring and defoaming predetermined constituent raw materials to form a surface layer, wherein said predetermined constituent raw materials and contents thereof include: 100 parts of resin, 5-30 parts of Maifan stone powder, 5-10 parts of quartz powder, 5-30 parts of tourmaline powder, and 1-5 parts of rare earth, wherein the mass part of the resin is taken as a standard; and
coating the surface layer on a release paper, sticking a polyurethane bass after drying, and performing a separation from the release paper after drying and cooling.

10. The method of making an active material according to claim 9, wherein said predetermined constituent raw materials and the contents thereof include: 100 parts of resin, 5-8 parts of Maifan stone powder, 5-8 parts of quartz powder, 5-10 parts of tourmaline powder, and 1-5 parts of rare earth, wherein the mass part of the resin is taken as a standard.
